(19) United States
(12) Patent Application Publication
(10) Pub. No.: US 2023/0277445 A1
(43) Pub. Date: Sep. 7, 2023

(54) ADHESIVE DRUG CARRIER

(71) Applicants: UMC Utrecht Holding B.V., Utrecht (NL); SentryX B.V., Austerlitz (NL)

(72) Inventors: Susanna Piluso, Utrecht (NL); Jasper Gerard Steverink, Austerlitz (NL); Floris Rudolf Van Tol, Austerlitz (NL); Raymond Michel Schiffelers, Utrecht (NL); Bas Jeroen Oosterman, Austerlitz (NL); Joannes Jacobus Verlaan, Utrecht (NL)

(73) Assignees: UMC Utrecht Holding B.V., Utrecht (NL); SentryX B.V., Austerlitz (NL)

(21) Appl. No.: 18/001,375

(22) PCT Filed: Jun. 10, 2021
(86) PCT No.: PCT/EP2021/065707

§ 371 Date: Dec. 9, 2022

(30) Foreign Application Priority Data
Jun. 11, 2020 (NL) 2025818

(51) Int. Cl. A61K 9/00; A61K 47/42; A61K 31/445; A61L 24/10; A61L 24/00

(52) U.S. Cl. CPC A61K 9/0024; A61K 31/445; A61K 47/42; A61L 24/0015; A61L 24/0031; A61L 24/104; A61L 2300/402; A61L 2300/418

(57) ABSTRACT

The present invention relates to an adhesive drug carrier comprising an injectable hydrogel comprising at least one medication, wherein the hydrogel comprises a (i) a protein-based polymer functionalized with a functionalization agent that is able to form guest-host interactions with oxidized P-cyclodextrin, cross-linked with (ii) an oxidized P-cyclodextrin (oβ-CD) as matrix and the at least one medication (iii), and wherein the hydrogel further comprises (iv) a protein-based polymer bearing quinone and/or catechol groups. The invention further relates to a method for its preparation and a method for treatment.

### TECHNICAL FIELD

This invention concerns an improved adhesive drug carrier. More specifically, the present invention concerns an improved adhesive drug carrier that is suitable for small molecules, anaesthetics in particular.

### BACKGROUND ART

An oxidized β-cyclodextrin-functionalized injectable gelatin hydrogel as a new platform for tissue adhesive hydrophobic drug delivery is known from RSC Adv 2017, u, 34053-34064, by Thi and Lee et al. The authors combined horseradish peroxidase (HRP)-mediated and Schiff base reactions to develop in situ forming bioadhesive hydrogels with fast gelation, tunable material properties, improved adhesiveness, excellent biocompatibility, and ease of use. Although the hydrophilic nature of hydrogels normally restricts hydrophobic drug loading, this hydrogel composition contained solutions to allow it use as a hydrophobic drug carrier. Following partial oxidation of β-cyclodextrin, (oβ-CD) the resulting aldehyde groups spontaneously reacted with amine groups on gelatin to form a Schiff base. The β-cyclodextrin cavity was subsequently used to host hydrophobic drugs. Simply blending the GTA and oβ-CD under oxidation with HRP/H₂O₂ resulted in the fast formation of GTA-oβ-CD hydrogels within a few seconds to several minutes. The authors found that (i) their adhesive and mechanical strength was reinforced by dual-crosslinking, including phenol-phenol and via the Schiff base reaction, and (ii) their hydrophobic drug loading efficacy was higher compared with pure GTA hydrogels. Adhesion, however, leaves still room for improvement. For instance, the gelatin hydrogel cannot be used for adhesion to bone or metal or for adhering a preformed gelatin hydrogel.

Mussels are known for their strong adherence to e.g. hulls of ships via byssal threads. Mussel foot proteins in these byssal threads have high 3,4-dihydroxy-L-phenylalanine (DOPA) content. Studies have shown that DOPA can strongly and reversibly bind to Titanium and also to wet bone and tissue. DOPA and catechol compounds (e.g. 3, 4-dihydroxyphenylacetic acid) are therefore interesting moieties to include in biomaterials for adhesion purposes.

As is known from Acta Biomaterialia 33 (2016) 51-63, Fan et al, medical adhesives for in-situ use need to meet very stringent conditions to avoid allergic reactions to patients. Fibrin glues (such as Tisseel), albumin-glutaraldehyde adhesives (such as BioGlue), and cyanoacrylates (such as Dermabond) are well-known and currently used in many surgical procedures. However, the utilization of fibrin glues involves risks of blood-borne disease transmission and allergic reactions to patients; the high toxicities of aldehyde-containing products severely limit in vivo applications of the relevant adhesive products. Fan et al therefore developed a gelatin-dopamine conjugate that was used for the fabrication of a genipin-crosslink tissue adhesive.

In WO 2019117715 a deformable body is disclosed that is an anaesthetic-carrying body intended to be pressed with its bone contact surface on the periosteum of an outer surface of a bone to be treated. It would be interesting to be able to adhere the anaesthetic-carrying body to the surgical implant. Adhesion of the anaesthetic-carrying body to surgical implants (such as, for instance, plates, screws, joint prostheses, rods, nail, etc.) would facilitate correct initial placement of the anaesthetic-carrying body during implantation. Moreover, it would be interesting to be able to adhere the anaesthetic-carrying body to the bone, without the need for screws and other surgical implants for fixation of the anaesthetic-carrying body. Similarly, it would be interesting to be able to adhere the anaesthetic-carrying body to soft tissues, to provide a local drug delivery platform. Furthermore, it would be interesting to be able to adhere the anaesthetic-carrying body to itself, e.g., to allow closing of a (for example, horse-shoe shaped) snap-on total hip replacement drug delivery ring.

From Journal of Controlled Release, 95 (2004), 391-402 the design of a novel hydrogel-based intelligent system for controlled drug release is known. This paper focuses on the design of an assembled drug delivery system (DDS) to provide multifunctions, such as drug protection, self-regulated oscillatory release, and targeted uni-directional delivery by a bi-layered self-folding gate and simple surface mucoadhesion. Controlled release of a small drug molecule is not well possible.

Of additional clinical relevance and interest would be an adhesive drug carrier that might be used to attach soft tissue to soft tissue, soft tissue to surgical implants, soft tissue to bone, and bone to bone where the primary function is as medical glue, or as a kit/sealant, with the controlled release of medication as a further benefit.

Biomater. Sci., 2016, 4, 1726-1730, “Preserving the adhesion of catechol-conjugated hydrogels by thiourea-quinone coupling”, describes catechol-functionalized gelatin and hydrogels based thereon. The teaching of this paper is limited to specific catechol-conjugated hydrogels, wherein the adhesiveness to inorganic surfaces is maintained by crosslinking chemistry at acidic pH. This paper is silent on controlled release of medication. Moreover, there is no suggestion whether and how this teaching of crosslinking chemistry at acidic pH may be used to improve the adhesiveness of other hydrogels, without adversely affecting such other hydrogels.

The controlled release of small-molecule drugs remains a challenge, as this process is mainly dependent on passive diffusion. In most cases, the size of the drug molecule is smaller than the mesh size of the hydrogel.

From Biomaterials, 39 (2015), 173-181, “Adhesive barrier/directional controlled release for cartilage repair by endogenous progenitor cell recruitment”, the use of an adhesive layer is known. This reference describes a hydrogel depot for directional release of therapeutic proteins encapsulated therein. This reference describes an adhesive gel patch (chitosan-catechol) on top of a fibrin gel. The adhesive layer, however, acts as a barrier. Finding an adhesive layer that does not affect the controlled release at the connecting surface is therefore problematic.

The need therefore remains for a hydrogel that may be used as platform for drug delivery, with improved adhesiveness. Moreover, this medical adhesive should be suitable for in vivo applications, with (improved) adhesiveness to soft tissue, bone and metal, and meet the regulatory safety criteria. Furthermore, this adhesive drug carrier should not be restricted in its release of medication even at the connecting surface(s) where it is attached to e.g., soft tissue or bone and the like.

### SUMMARY OF THE INVENTION

The present invention provides an adhesive drug carrier comprising an injectable hydrogel comprising at least one medication, wherein the hydrogel comprises a (i) a protein-based polymer functionalized with a functionalization agent that is able to form guest-host interactions with oxidized β-cyclodextrin, cross-linked with (ii) an oxidized β-cyclodextrin (oβ-CD) as matrix and the at least one medication (iii), and wherein the hydrogel further comprises (iv) a protein-based polymer bearing quinone and/or catechol groups.

The adhesive drug carrier may comprise a medicament, preferably a small drug molecule with a molecular weight of less than 1000 Daltons, preferably a pain-relieving medicament, more preferably a local anesthetic, even more preferably bupivacaine.

The invention further relates to a method for its preparation, which allows the components to be injected into tissue or applied onto tissue, bone or surgical implants and then to cross-link.

One of the components used in the adhesive drug carrier of the present invention is believed to be novel and may be used as medical adhesive by itself. The invention therefore also relates to this medical adhesive, as well as its use.

### DRAWINGS

FIG. 1, a schematic representation of the adhesive drug carrier of the present invention glued onto or adjacent to a surgical plate.

FIG. 2, a schematic representation of application in a high tibial osteotomy procedure.

FIG. 3, a schematic representation of the adhesive drug carrier of the present invention glued onto soft tissue.

The abovefigures are for illustration purposes only and not necessarily to scale.

### DETAILED DESCRIPTION OF THE INVENTION

Hydrogels may be synthesized by cross-linking water-soluble polymers. The present invention focusses on medical hydrogels that are based on protein-based polymers. These hydrogels are biocompatible and can be implanted or injected and used in-vivo. Moreover, they are biodegradable, i.e. can be decomposed naturally inside the human body. Preferably, the protein-based polymer, is selected from commercially available biocompatible polymers that comprise amino and carboxylic groups, such as silk, fibrin, collagen or gelatin. More preferably, the hydrogel used in the present invention is based on gelatin. The hydrogel may also comprise other biocompatible water-soluble synthetic or natural polymers. The other polymers may compose up to 50% by weight on the entire polymer content. Given its availability, biocompatibility and cost, the use of gelatin as sole polymer component is preferred.

The protein-based polymer (i), preferably gelatin, is preferably functionalized with tyramine, 4-(2-amino-ethyl)phenol as functionalization agent. In addition to or instead of tyramine, other primary aminoalkylphenols of the formula NH₂-R-PhOH and substituted versions thereof may be used. Tyramine is the most commonly used compound to introduce phenolic hydroxyl groups on the gelatin backbone via functionalization with gelatin carboxylic acid groups. Alternatively, phenolic hydroxyl groups can be introduced via reaction with gelatin amino groups using functionalization agents such as hydroxyphenyl propionic acid, e.g., 3-(4-hydroxyphenyl)propionic acid, also known as desaminotyrosine . Of importance is the biocompatibility of the functionalization agent and its possibility to form guest-host interactions with the oxidized β-cyclodextrin. Given its availability, biocompatibility and cost, the use of tyramine as sole agent to functionalize the protein-based polymer is preferred.

The protein-based polymer (iv), preferably gelatin, is preferably created by reacting the protein-based polymer with a reactant bearing a catechol or quinone group or a derivative thereof. For instance, the protein-based polymer may be modified with catechol-containing compounds or a compound that can be easily oxidized to provide quinone-groups such as 2-(3,4-dihydroxyphenyl)ethylamine hydrochloride (dopamine), 3,4-dihydroxy-L-phenylalanine (DOPA), 3-(3,4-dihydroxyphenyl)-2-propenoic acid (CA, caffeic acid), or 3-(3,4-dihydroxyphenyl)propanoic acid (DHC, dihydrocaffeic acid), 3,4,5-trihydroxybenzoic acid (gallic acid), (R)-4-(1-hydroxy-2-(methylamino)ethyl)-1,2-benzenediol (epinephrine), or (R)-4-(2-amino-1-hydroxyethyl)-1,2-benzeendiol (norepinephrine). Moreover, the catechol-containing compounds may be partly oxidized into quinone-groups, or the quinone-groups may be partly reduced into catechol groups, thereby creating dual functionality. This may be of interest to achieve adhesion to e.g., soft tissue (quinone) together with adhesion to metals and other materials used in surgical implants (catechol). Derivatives of e.g., dopamine, DOPA, CA or DHC, with the functional group being temporarily protected, may also be used. Of importance is the biocompatibility of the reactant to avoid any toxicity issues. Given its availability, biocompatibility and cost, the use of DOPA, CA, DHCA or the protected derivatives thereof are preferred.

A significant amount of the amino groups of the protein-based polymer (iv) are functionalized. Preferably the protein-based polymer (iv), preferably gelatin, bears from 15% to 70% of the total gelatin amino groups, preferably from 20 to 50% of combined catechol and/or quinone groups per molecule. Below 15% of the gelatin amino groups, the adhesive performance is too low when using regular amounts of polymer (iv). Above 70% of the gelatin amino groups, achieving a homogeneous injectable hydrogel becomes problematic. The protein-based polymer preferably has a molecular weight in the range of 50 to 200 kDa, preferably 90 to 150 kDa. Below 50 kDa the interaction with the hydrogel (cohesion) is insufficient. Above 200 kDa achieving a homogeneous injectable hydrogel becomes problematic.

As indicated above, the adhesive drug carrier may be created by forming a hydrogel using a combination of the protein-based polymers (i) and (iv).

The amount of oxidized β-cyclodextrin versus the combination of the protein-based polymers (i) and (iv), preferably gelatin, may vary within broad ranges. Preferably, the amount of oβ-CD may be from 0.1% to 10% by weight of the hydrogels, preferably in the range of 2% to 6% by weight of the hydrogel. Using a higher amount of oxidized β-cyclodextrin can interfere with the chemical crosslinking of the protein-based polymer, due to increasing interaction between the tyramine functional groups and the oxidized β-cyclodextrin cavity.

The combination of the protein-based polymers (i) and (iv) may be used in very broad ranges. For instance, they may be used in a weight ratio of 9:1 to 1:9, preferably 7:3 to 3:7, more preferably 3:2 to 2:3, most preferably about 1:1. Using a higher amount of (i) may adversely affect the adhesive properties of the adhesive drug carrier. Using a higher amount of (iv) may adversely affect the cross-linking density and therefore cohesive strength of the hydrogel.

The use of β-cyclodextrin in hydrogels is known. In the present invention, the β-cyclodextrin is oxidized. The oxidation of β-cyclodextrin is needed to enable the grafting to the protein-based polymer. The degree of oxidation may vary from 5-40%, preferably from 20-30% of the secondary hydroxyl groups. Oxidation results in the conversion of secondary hydroxyl groups in the molecule into aldehyde groups. The preferred oxidation degree allows for maximal grafting of oβ-CD on the gelatin backbone, while limiting the cytotoxic effects that may results from any unreacted aldehyde groups and ensuring sufficient solubility of oβ-CD in water.

Ideally the adhesive drug carrier is able to release medication in a controlled manner. For small size molecules, with a molecular weight below 1000 Daltons, like e.g. bupivacaine, this is particularly challenging. Accordingly, it is preferred that a particular cross-link density is achieved, resulting in a degree of swelling that is in the range of 2-20, preferably in the range of 2-6, calculated as swollen weight (at equilibrium swelling) - dry weight/dry weight. The swollen weight is the equilibrium weight of the hydrogel in vivo. The swollen weight may be experimentally determined in vitro after 24h of swelling (or when it reaches equilibrium) in a simulated body fluid such as PBS at body temperature, such as 37° C. The cross-link density is achieved by using crosslinks of the types:
(a) phenol-phenol crosslinks in the protein-based polymer (i) functionalized with a primary aminoalkylphenol or similar functionalization agent, and phenol-phenol crosslinks in the protein-based polymer (iv) bearing a catechol or quinone group,
(b) Schiff-base crosslinks between amino groups present on the functionalized protein-based polymer and aldehyde groups of oβ-CD, and
(c) guest-host interactions between phenol moieties of the functionalization agent grafted on the protein-based polymer and the cavity of oβ-CD.

The present invention in particular provides excellent control and adjustability of the formation of the phenol-phenol crosslinks. As a result, hydrogels may be produced with a wide variety of ratios between the crosslink types (a), (b) and (c). Moreover, by attuning the crosslink density, also the elasticity can be varied. The relevance of this is discussed herein below, where the various embodiments of the hydrogels of the present invention are discussed.

In the preparation of the hydrogel, a cross-linking agent or (photo)initiator may be used, but need not be used to form crosslinks. Use of a cross-linking agent or (photo)initiator is not essential to form a hydrogel, as some of the crosslinks mentioned above may occur spontaneously by mixing the components at a suitable temperature.

Crosslinking systems for cross-links of type (a) are known in the art. For instance, they may be based on HRP/H₂O₂ or similar system. Crosslinks may also be created by use of a photoinitiator, for instance by use of a combination of riboflavin, sodium persulfate (SPS) and visible light. Riboflavin, also known as vitamin B2, is naturally recurring in the body, is biocompatible and is currently used in clinical application for the crosslinking of corneal collagen (Belin, Michael W., et al. Cornea 2018, 37,1218-1225). The exposure of riboflavin to visible light, in presence of SPS, generates reactive intermediates. With visible light, the portion of the electromagnetic spectrum that is visible to the human eye is meant. A typical human eye will respond to wavelengths from about 380 to about 740 or even 780 nanometers. In particular, the invention has been tested with wavelengths between 400 and 700 nanometers. Other useable photo initiators would be ferrocene, and anthraquinone.

If used, then the use of a photo-induced crosslinking provides for better control and adjustability than the HRP/H₂O₂ system known from the prior art. Use of a photoinitiator is therefore preferred. For instance, the riboflavin and SPS may be used in a molar ratio of 1:5-20, preferably about 1:10 (riboflavin:SPS). For instance, riboflavin and SPS may be used at 0.1-10 mM for riboflavin and 1-100 mM for SPS. Preferably the riboflavin, if used, is flavin mononucleotide, which is the water-soluble form of riboflavin.

In a co-pending application, the use of a hydrogel as anaesthetic-carrying body for local release of medication in the form of a ring is described (WO2019117715, incorporated herein by reference) where it is used in combination with a screw. In an alternative embodiment, the anaesthetic-carrying body is glued on a plate element. Information on a suitable manner to glue the anaesthetic-carrying body onto a plate element is not provided. By providing an injectable hydrogel as carrier for local release of medication, the need for a screw or similar form of attachment is avoided.

In addition to the applications identified above, the adhesive drug carrier may be injected/inserted as mixture of components in a viscous form, where after it will cross-link in-situ. In this manner, it can be applied onto irregular or tight anatomical spaces, without the need for a predefined form. After in-situ cross linking, it may still be pliable so that no significant movement restrictions will occur. It may also be used to adhere soft tissue to soft tissue. For instance, it may be applied as a topical skin adhesive where it reacts spontaneously upon contact with weak bases such as water, blood or cell membranes, to assist in wound closure. It may also be used to adhere soft tissues inside the body to repair for instance tissue lacerations, for instance, blood vessels or bowel. It may similarly be used to attach soft tissue to surgical implants. For instance, it may attach tendons to a surgical plate after fracture reduction, e.g., where such tendons are either traumatically/degeneratively torn or had to be surgically released during exposure of the fracture. Moreover, the adhesive drug carrier may be used to aid in fixation of fractures, whereby bone fragments are attached to each other or to other surgical elements. Components advancing the healing process or strengthening the juncture formed by the adhesive drug may be added. Moreover, it may be used to attach soft tissue and/or bone to metal. Existing hydrogels may be attached in a similar manner.

The adhesive drug carrier may be applied in a living human or animal body. Each of these embodiments is very suitable for treatment of medical disorders such as musculoskeletal disorders, and especially for treatment of skeletal disorders due to the ability of the hydrogel to adapt to the shape of a bone, tissue or surgical implant to which it has been pressed. This ability is of interest also in other applications where its presence if it would lack flexibility would cause hindrance to the patient and/or restrict movement. These disorders include infection, inflammation, auto-immune disease, malignant and benign neoplasms, growth disorders, trauma, degenerative disorders or treatment of pain arising from (surgical treatment of) these disorders.

The present invention is described with reference to the use of bupivacaine, but any (local) anaesthetic may be used. Local anaesthetics are typically divided into amides and esters; amides are more commonly used. The anaesthetic is preferably an amino-amide local anaesthetic, such as articaine, etidocaine, prilocaine, bupivacaine, levobupivacaine, ropivacaine, mepivacaine, lidocaine, dibucaine, or other amino-caine, but may also be ester-based, such as tetracaine, procaine or chloroprocaine. The anaesthetic may also comprise a combination of two or more types of anaesthetics. Preferably, the anaesthetic is bupivacaine, liposome bupivacaine or levobupivacaine, lidocaine, or a combination of anaesthetics comprising bupivacaine, liposome bupivacaine and/or levobupivacaine. The medication may also be or comprise an antibiotic or anticancer agent, a growth factor, an immunomodulatory drug, chemotherapeutic agents, steroids (including retinoids), hormones, anti-microbials, antivirals, anti-inflammatory compounds, radiation absorbers, including UV-absorbers, radiation enhancers, a hemostatic agent, vaccines, stem cells, etc. The medication may furthermore be hydrophilic or hydrophobic. Due to the hydrophilic nature of the hydrogel, a hydrophilic medication is easily incorporated into the hydrogel. The hydrophobic cavity of oβ-CD provides encapsulation for hydrophobic drugs. Therefore, with respect to hydrophobic medication, the hydrogel of the present invention has an advantage over hydrogels not comprising oβ-CD. Preferably, the medication is hydrophobic. A measure for the hydrophobicity of a medication is the octanol-water-partition coefficient P, which is the ratio of the concentrations of the medication in a mixture of octanol and water at equilibrium. For a hydrophobic medication log P > 0, preferably log P > 2.

Controlled release of bupivacaine and similar small drug molecules with a molecular weight smaller than 1000 Daltons is challenging, but can be achieved with the hydrogel of the present invention. Moreover, directional release may be further improved by providing a barrier layer e.g., on the surface of the adhesive drug carrier opposite to the surface where it is connected and intended to release the medication.

The hydrogel may comprise additional components, like colorants, stabilizers, co-solvents, buffers and similar common additives. If and to the extent that bupivacaine is used as medication, it is preferably used in an amount of 0.01-200 mg/mL volume. Moreover, the medication may itself be encapsulated in nano- or microparticles, for instance, at a size range of 50 nm to 200 µm, prior to its inclusion in the hydrogel. It may be encapsulated in PLGA, PCL, gelatin, alginate or liposomes.

The medication may be added while dissolved in a co-solvent, or added in the form of crystals.

In addition to the medication one or more further ingredients may be included, preferably further ingredients selected from co-medication, co-solvents, surfactants, colorants, and buffers. Co-medication may be considered to be any further medication added to the hydrogel, preferably medication which enhances the effect of the at least one medication present in the hydrogel and/or advances the healing process. Co-solvents include but are not limited to plasticizers. One such plasticizer is glycerol. The addition of glycerol into the hydrogel matrix results in higher elasticity, yet does not affect sample stiffness. Other co-solvents may be chosen for their ability to improve drug solubility during loading, such as DMSO or ethanol.

Methods for making the feedstock for the hydrogel are known. Thus, it is known to functionalize gelatin and related protein-based polymers with tyramine and related primary aminoalkylphenols. Moreover, gelatin desaminotyrosine (“GelDat”) is commercially available. Likewise, it is known to oxidize β cyclodextrin. See Thi et al, RSC Adv. 2017, which has been cited above, and which is included herein by reference. Of importance, but common in the field of medical application is to remove all forms of contamination. By way of example, the hydrogel may be prepared by the following method:

1. Solutions of (i), (ii), (iii) (iv) and optional initiators are prepared.

2. These solutions, are mixed so that pre-determined concentrations are obtained.

These concentrations can be varied depending on the desired mechanical and release properties.

3. The obtained solution is then allowed to cross-link. For instance, it may be injected into tissue, where it is allowed to cross-link.

The injectable hydrogel may be applied with a devise that mixes the components upon use. For instance a dual barrel syringe may be used, connected to a mixing tip. In this case the injectable hydrogel would be used in the form of a kit of components. For instance, one syringe would contain (iv) and the other the remaining components.

In this case, the injectable hydrogel can be injected directly from the mixing tip to a tissue or metal plate. As an alternative the mixing tip can be connected to a mould with an open base. This way the mixture will be injected via the mould, adhering on the tissue and adopting the shape of the mould. After few minutes the mould can be removed.

By way of example, the solution containing (iv) may be prepared as follows:

### Recipe 1 Catechol or Quinone Functionality)

In a first step gelatin with a Mw of 120 kDa was functionalized with DHC or CA in an amount of 2,5, 5 or 10%wt respectively. Various routes are available:
example of functionalization with CA: EDC/NHS is used to activate the carboxylic group of CA and then reacted with the gelatin lysine amino groups at pH of about 4.5-5. The gelatin-CA (GCA) conjugate is then purified in an acidic environment (e.g. dialysis against HCl solution) to prevent the oxidation to quinone.
example of functionalization with DHC: DHC is activated with EDC/NHS in MES buffer (pH 4.5-5) and then reacted with gelatin lysine amino groups. Purification of the gelatin-DHC conjugate (GDHC) is similar to previous.

For quinone functionality, the GCA or GDCH is dissolved in an alkaline solution (e.g. pH 8-8.5) to promote the oxidation of the catechol groups to quinone. Quinone may also be formed by the addition of an oxidizing agent, e.g., SPS or H₂O₂.

### Recipe 2 (Quinone and Catechol Functionality)

The double functionality may for instance be obtained by mixing GCA and GDHC, followed by oxidation. When using mild conditions, the formation of quinones will only occur for GCA.

### Example 1, Injectable Hydrogel, Adhesion Test

A protein-based polymer (i) functionalized with a hydroxyphenyl propionic acid as functionalization agent, (10 w%), was mixed with varying ratios of GCA and/or GDHC, (iv), with a degree of functionalization of about 15%, respectively about 25%, to a maximum of 10 w% of the total volume in water and put overnight in an incubator at 45° C.

The next day oβ-CD (8 w%) and an oxidizing/crosslinking agent (e.g. 5 mM SPS or 0.3 w% H₂O₂) was added. The adhesion test was performed without medication, but this may be added at this stage too.

After mixing, 100 µL of the injectable was put on a titanium slice (“Ti”) or piece of tenderloin (“tissue”) and another titanium slice/tissue was pressed on top of it (during ± 2 seconds). The adhesion was assessed. Whether the injectable hydrogel was adhesive, was tested by pulling the two surfaces from each other. The injectable hydrogels 1-6 were compared with a hydrogel C, containing neither GCA nor GDHC, thus illustrating the prior art. The results are summarized in Table 1.

TABLE 1

| | 5 mM SPS | | | | 0.3 w% H₂O₂ | | | |
|---|---|---|---|---|---|---|---|---|
| | C | 1 | 2 | 3 | C | 1 | 2 | 3 |
| GDHC (%) | 0 | 10 | | 5 | 0 | 5 | | 2,5 |
| GCA (%) | 0 | | 10 | 5 | 0 | | 5 | 2,5 |
| Ti-Ti | X | V | V | V | X | V | V | V |
| Ti-tissue | X | V | V | V | X | V | V | V |
| tissue-tissue | X | V | V | V | X | V | V | V |

As can be seen, the hydrogel C without GDHC or GCA, failed in the adhesion test. The injectable hydrogel containing GDHC and/or GCA sticks Ti-Ti, Ti-tissue and tissue-tissue. Moreover, the injectable hydrogel containing both GCA and GDHC showed increased cohesiveness as compared to injectable hydrogels containing either GCA or GDHC, when submerged 4 days in water. In addition, the hydrogel containing both GCA and GDHC provided adhesive strength much faster than the hydrogels containing only GCA or GDHC.

### Example 2, Injectable Hydrogel With Medication

Example 1 (with hydrogels C, 1 to 3) was repeated, but now with addition of 2,5 w% bupivacaine (“bupi”). The adhesiveness was not affected.

TABLE 2

| | C | 1 | 2 | 3 |
|---|---|---|---|---|
| Bupi (%) | 2,5 | 2,5 | 2,5 | 2,5 |
| GDHC (%) | 0 | 10 | | 5 |
| GCA (%) | 0 | | 10 | 5 |
| Ti-Ti | X | V | V | V |
| Ti-tissue | X | V | V | V |
| tissue-tissue | X | V | V | V |

### General Applicability

Embodiment of the invention will now be described, by way of example only, with reference to the accompanying schematic drawings.

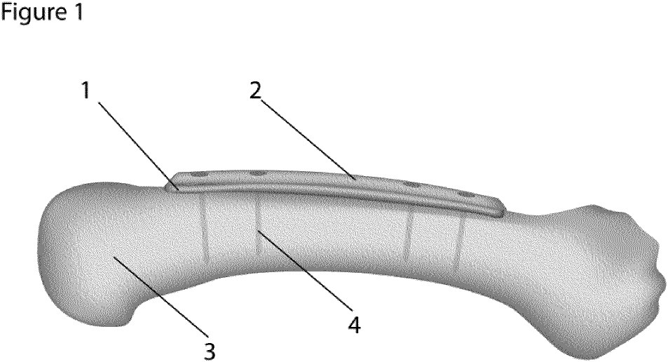

FIG. 1 shows a schematic representation of the adhesive drug carrier of the present invention (1) glued onto or beneath a surgical plate (2) which is attached to the bone (3) via surgical screws (4).

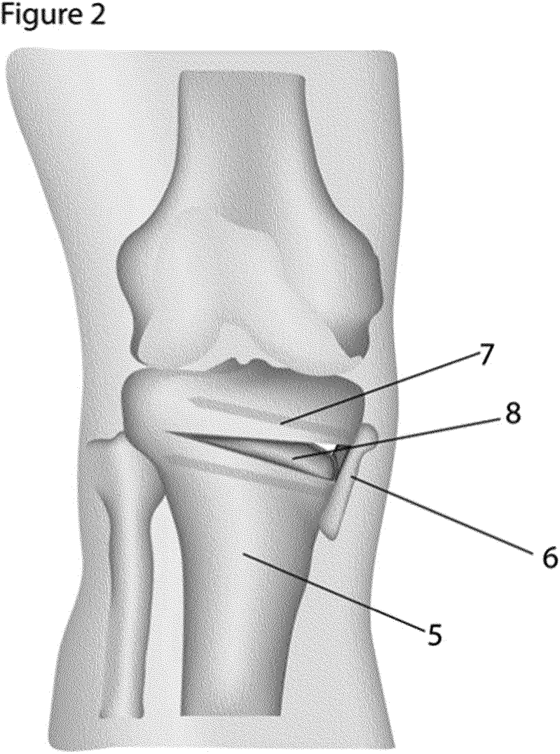

FIG. 2 shows a schematic representation of a high tibial osteotomy. The femur (5) is wedged open after which the open wedge is supported by a surgical plate (6) which is attached to the bone (5) via surgical screws (7). In the open wedge the drug carrier of the present invention (8) is attached onto the bone (5).

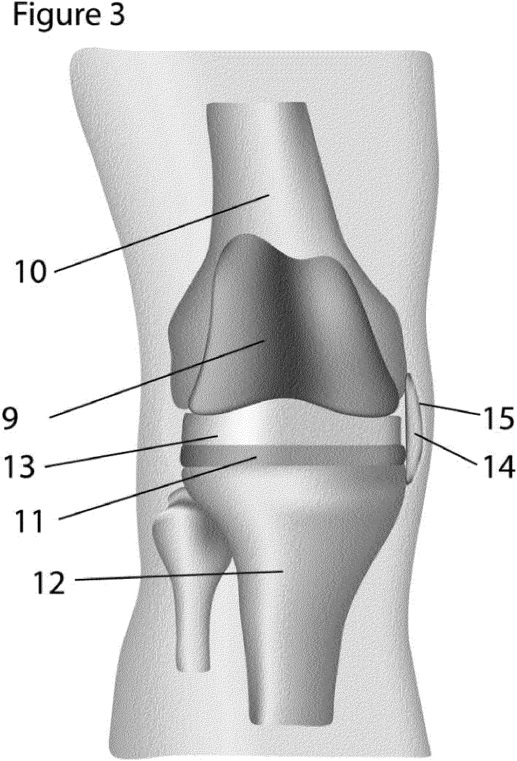

FIG. 3 shows a schematic representation of a total knee replacement. A femoral component (9) is attached to the femur (10) and a tibial component (11) is attached to the tibia (12) of the patient. The tibial component may be provided with a liner (13). The adhesive drug carrier of the current invention (14) is fixated onto the joint capsule (15), for instance in the medial or lateral gutter, infrapatellar, or may be attached cranially to the femoral component in the suprapatellar recess or to the liner located between the metallic components.

1. An adhesive drug carrier comprising an injectable hydrogel comprising at least one medication, wherein the hydrogel comprises (i) a protein-based polymer functionalized with a functionalization agent that is able to form guest-host interactions with oxidized β-cyclodextrin, cross-linked with (ii) an oxidized β-cyclodextrin (oβ-CD) as matrix and the at least one medication (iii), and wherein the hydrogel further comprises (iv) a protein-based polymer bearing quinone and/or catechol groups, wherein
the protein-based polymer (iv) has a molecular weight in the range of 50 to 200 kDa and bears from 15% to 70% of the total amino groups of combined catechol and/or quinone groups per molecule;
the protein-base polymer (i) and the protein-based polymer (iv) are selected from silk, fibrin, collagen or gelatin;
the amount of oβ-CD is from 0.1 to 10% by weight of the hydrogel, and
the at least one medication (iii) is a small drug molecule with a molecular weight of less than 1000 Daltons.

2. The adhesive drug carrier according to claim 1, wherein the hydrogel comprises (iv) a protein-based polymer bearing quinone groups and a protein-based polymer bearing catechol groups.

3. The adhesive drug carrier according to claim 2, wherein the hydrogel comprises a cross-linked mixture of (i), (iv) and (ii), with (i) and (iv) in a weight ratio selected from a group consisting of: 9:1 to 1:9, 7:3 to 3:7, 3:2 to 2:3, about 1: 1.

4. The adhesive drug carrier according to claim 1, wherein the protein-based polymer (i) and the protein based polymer (iv) are the same.

5. The adhesive drug carrier according to claim 1, wherein the protein-based polymer (i) is functionalized with a primary aminoalkylphenol or with a hydroxyphenyl propionic acid.

6. The adhesive drug carrier according to claim 1, wherein the protein-based polymer (iv) is made by modification with 2-(3,4-dihydroxyphenyl)ethylamine hydrochloride (dopamine), 3,4-dihydroxy-L-phenylalanine (DOPA), 3-(3,4-dihydroxyphenyl)-2-propenoic acid (CA, caffeic acid), 3-(3,4-dihydroxyphenyl)propanoic acid (DHC, dihydrocaffeic acid), 3,4,5-trihydroxybenzoic acid (gallic acid), (R)-4-(1-hydroxy-2-(methylamino)ethyl)-1,2-benzenediol (epinephrine), or (R)-4-(2-amino-1-hydroxyethyl)-1,2-benzeendiol (norepinephrine).

7. The adhesive drug carrier according to claim 1, wherein the medication (iii) is selected from the group including: a hydrophobic medication, a pain-relieving medicament, a local anesthetic, and bupivacaine.

8. The adhesive drug carrier according to claim 1, for use in the treatment of medical disorders, wherein the hydrogel is adhered to itself, bone, tissue or surgical metal by way of the protein-based polymer bearing quinone and/or catechol groups.

9. The adhesive drug carrier according to claim 1, for use as hemostatic or for the delivery of antigens, nucleic acids-encoding agents or antigen-encoding viral vectors, wherein the hydrogel is adhered to itself, bone, tissue or surgical metal by way of the protein-based polymer bearing quinone and/or catechol groups.

10. The adhesive drug carrier according to claim 1, as a kit of parts.

11. A method for preparing the adhesive drug carrier according to claim 1, wherein (iv) is part of the cross-linked hydrogel, the method comprising:
preparing a mixed solution of (i) the protein-based polymer functionalized with functionalization agent, (ii) oβ-CD, (iii) the medication, (iv), the protein-based polymer bearing quinone and/or catechol groups, and
crosslinking the hydrogel.

12. The method according to claim 11, wherein the mixed solution of (i), (ii), (iii) and (iv) is introduced by injection into or applied onto a patient prior to crosslinking.

13. A method for treatment of medical disorders, comprising the localized application of the adhesive drug carrier according to claim 1.

14. A method for use as hemostatic or for the delivery of antigens or antigen-coding viral vectors, comprising the localized application of the adhesive drug carrier according to claim 1.

15. The adhesive drug carrier according to claim 4, wherein the protein-based polymer (i) and the protein based polymer (iv) are the same, being gelatin.

16. The adhesive drug carrier according to claim 5, wherein the protein-based polymer (i) is functionalized with tyramine or with 3-(4-hydroxyphenyl)propionic acid.

17. The adhesive drug carrier according to claim 5, wherein the protein-based polymer (i) is gelatin functionalized with tyramine (GTA) or gelatin functionalized with desaminotyrosine.

18. The adhesive drug carrier according to claim 1, for use in the treatment of a disorder selected from the group including: musculoskeletal disorders, infection, inflammation, auto-immune disease, malignant and benign neoplasms, growth disorders, trauma and degenerative disorders, or treatment of pain arising from surgical treatment of these disorders, wherein the hydrogel is adhered to itself, bone, tissue or surgical metal by way of the protein-based polymer bearing quinone and/or catechol groups.

19. The adhesive drug carrier according claim 10, as a kit of parts, for use in a dual barrel syringe.

20. A method for treatment of a disorder selected from the group including: musculoskeletal disorders, infection, inflammation, auto-immune disease, malignant and benign neoplasms, growth disorders, trauma and degenerative disorders, or treatment of pain arising from surgical treatment of these disorders, comprising the localized application of the adhesive drug carrier according to claim 1.
